(19) United States
(12) Patent Application Publication
(10) Pub. No.: US 2023/0010317 A1
(43) Pub. Date: Jan. 12, 2023

(54) MEDICAL IMAGE PROCESSING DEVICE AND OPERATION METHOD THEREOF

(71) Applicant: FUJIFILM Corporation, Tokyo (JP)

(72) Inventor: Misaki GOTO, Tokyo (JP)

(73) Assignee: FUJIFILM Corporation, Tokyo (JP)

(21) Appl. No.: 17/811,459

(22) Filed: Jul. 8, 2022

(30) Foreign Application Priority Data
Jul. 12, 2021 (JP) 2021-115081

(51) Int. Cl. G16H 30/40; G06V 20/40; G06V 10/60; G06V 10/26; G06V 10/771

(52) U.S. Cl. CPC G16H 30/40; G06V 20/46; G06V 10/60; G06V 10/26; G06V 10/771

(57) ABSTRACT

A medical image processing device includes an image acquisition unit that acquires a medical video image, a brightness analysis unit that analyzes brightness information of each of a plurality of specific medical images within a specific time range among a plurality of medical images constituting the medical video image to output brightness analysis information, and an image selection unit that selects a training medical image to be used for machine learning from among the plurality of specific medical images using the brightness analysis information.

### CROSS-REFERENCE TO RELATED APPLICATIONS

This application claims priority under 35 U.S.C § 119(a) to Japanese Patent Application No. 2021-115081 filed on 12 Jul. 2021. The above application is hereby expressly incorporated by reference, in its entirety, into the present application.

### BACKGROUND OF THE INVENTION

### 1. Field of the Invention

The present invention relates to a medical image processing device and an operation method thereof.

### 2. Description of the Related Art

In a medical field, a medical image, such as an endoscopic image, an X-ray image, a computed tomography (CT) image, and a magnetic resonance (MR) image, is used to perform image diagnosis, such as diagnosis of a medical condition of a patient or follow-up. Based on such image diagnosis, a doctor and the like determine treatment policies.

In recent years, machine learning, such as deep learning, has become widespread in the image diagnosis using the medical image. WO2019/198637A (corresponding to US2021/012495A1) discloses a medical image processing device that acquires a medical video image composed of a plurality of medical images and report information corresponding to the medical video image, analyzes the report information, and stores the medical image including a lesion image in a storage device. The medical image stored in the storage device is used for machine learning.

On the other hand, as an image diagnosis system using machine learning, a computer aided diagnosis (hereinafter, referred to as CAD) that analyzes a medical image, such as an endoscopic image, to automatically detect a region of interest, such as a lesion, and highlights the detected region of interest is known.

### SUMMARY OF THE INVENTION

In order to develop the CAD using machine learning, it is necessary to acquire a large amount of medical images. Therefore, it is considered to perform machine learning using the medical video image captured during an examination. The medical video image includes the plurality of medical images captured in a time series manner.

However, the medical video image includes a medical image with a low image quality captured in a state in which blurriness, blur, or water fog occurs, or in a state of being in close contact with a mucous membrane. These medical images with a low image quality are not suitable for machine learning for the development of the CAD. Therefore, in order to perform machine learning using the medical video image, it is necessary to exclude the medical video image with a low image quality. However, the medical image processing device disclosed in WO2019/198637A does not have a configuration for excluding the medical image with a low image quality from the medical video image. Therefore, it is necessary to display the plurality of medical images on a display or the like, and a user needs to visually confirm and exclude the medical images having a low image quality, which takes labor and time.

The present invention is to provide a medical image processing device capable of efficiently selecting a medical image suitable for machine learning from a medical video image and an operation method thereof.

An aspect of the present invention relates to a medical image processing device comprising a processor, in which the processor acquires a medical video image, analyzes brightness information of each of a plurality of specific medical images within a specific time range among a plurality of medical images constituting the medical video image to output brightness analysis information, and selects a training medical image to be used for machine learning from among the plurality of specific medical images using the brightness analysis information.

It is preferable that the processor outputs, as the brightness analysis information, size information of a region having brightness equal to or greater than a certain threshold value for each of the plurality of specific medical images, and compares the size information of the plurality of specific medical images to select the training medical image to be used for machine learning from among the plurality of specific medical images.

It is preferable that the processor output, as the brightness analysis information, size information and positional information of a region having brightness equal to or greater than a certain threshold value for each of the plurality of specific medical images, and compare the size information and the positional information of the plurality of specific medical images to select the training medical image to be used for machine learning from among the plurality of specific medical images.

It is preferable that the processor calculate the size information for a target medical image and a peripheral medical image different from the target medical image among the plurality of medical images constituting the medical video image, calculate a change amount of the size information between the target medical image and the peripheral medical image, and determine whether or not to select the target medical image as the training medical image using the change amount.

It is preferable that the processor calculate the size information for a target medical image and a plurality of peripheral medical images different from the target medical image among the plurality of medical images constituting the medical video image, calculate a difference of a change amount using the size information of the target medical image and the plurality of peripheral medical images, and determine whether or not to select the target medical image as the training medical image using the difference.

It is preferable that the processor calculate a first pixel number which is the number of pixels of the region having the brightness equal to or greater than the certain threshold value for a target medical image among the plurality of specific medical images, calculate a second pixel number which is the number of pixels of the region having the brightness equal to or greater than the certain threshold value for a peripheral medical image different from the target medical image among the plurality of specific medical images, output the first pixel number and the second pixel number as the brightness analysis information, and determine whether or not to select the target medical image as the training medical image by comparing the first pixel number with the second pixel number.

It is preferable that the processor exclude the target medical image from the training medical image in a case in which the first pixel number is greater than a maximum value, an average value, or a median value of the second pixel number.

It is preferable that the processor divide a target medical image among the plurality of specific medical images into a plurality of regions, output, as the brightness analysis information, divided region brightness analysis information which is size information of a region having brightness equal to or greater than a certain threshold value for each of the plurality of regions of the target medical image, calculate peripheral brightness analysis information which is size information of the region having the brightness equal to or greater than the certain threshold value for a peripheral medical image different from the target medical image, and compare the divided region brightness analysis information with the peripheral brightness analysis information to select the training medical image to be used for machine learning from among the plurality of specific medical images.

It is preferable that the processor divide a target medical image among the plurality of specific medical images into a plurality of regions, output, as the brightness analysis information, divided region brightness analysis information which is size information and positional information of a region having brightness equal to or greater than a certain threshold value for each of the plurality of regions of the target medical image, calculate peripheral brightness analysis information which is size information and positional information of the region having the brightness equal to or greater than the certain threshold value for a peripheral medical image different from the target medical image, and compare the divided region brightness analysis information with the peripheral brightness analysis information to select the training medical image to be used for machine learning from among the plurality of specific medical images.

It is preferable that the processor divide a target medical image among the plurality of specific medical images into a plurality of regions, calculate a second pixel number which is the number of pixels of a region having brightness equal to or greater than a certain threshold value for a peripheral medical image different from the target medical image among the plurality of specific medical images, calculate a third pixel number which is the number of pixels of the region having the brightness equal to or greater than the certain threshold value for each of the plurality of regions, output the second pixel number and the third pixel number as the brightness analysis information, extract, as a target region, the region having the brightness equal to or greater than the certain threshold value among the plurality of regions by comparing the second pixel number with the third pixel number in at least one of the plurality of regions, and determine whether or not to select the target medical image as the training medical image by comparing the target medical image with the target region.

It is preferable that the processor use, as the target region, the region having the brightness equal to or greater than the certain threshold value in the plurality of regions corresponding to the third pixel number in a case in which the third pixel number is greater than a maximum value, an average value, or a median value of the second pixel number.

It is preferable that the processor exclude the target medical image from the training medical image in a case in which a total number of pixels of the target region is equal to or greater than a certain ratio of the number of pixels of the target medical image.

It is preferable that the processor exclude the target medical image from the training medical image in a case in which a maximum value, an average value, or a median value of the number of pixels of the target region is equal to or greater than a certain ratio of the number of pixels of the target medical image.

It is preferable that the processor determine the certain threshold value used in a case of outputting the brightness analysis information in accordance with operation information, an imaging condition, or an imaging apparatus in a case of capturing the medical image.

It is preferable that the processor analyze the brightness analysis information from three or more specific medical images to select the training medical image.

It is preferable that the processor store the training medical image in a storage device. It is preferable that the processor perform machine learning using the selected training medical image.

Another aspect of the present invention relates to an operation method of a medical image processing device, the method comprising a step of acquiring a medical video image, a step of analyzing brightness information of each of a plurality of specific medical images within a specific time range among a plurality of medical images constituting the medical video image to output brightness analysis information, and a step of selecting a training medical image to be used for machine learning from among the plurality of specific medical images using the brightness analysis information.

According to the present invention, it is possible to efficiently select the medical image suitable for machine learning from the medical video image.

### BRIEF DESCRIPTION OF THE DRAWINGS

FIG. 1 is a block diagram showing functions of a medical image processing system and an endoscope system.

FIG. 2 is a block diagram showing a function of a medical image processing device.

FIG. 3 is an explanatory diagram showing brightness analysis processing and selection processing.

FIG. 4 is a flowchart relating to selection of a training endoscopic image according to a first embodiment.

FIG. 5 is an explanatory diagram showing brightness analysis processing and selection processing according to a second embodiment.

FIG. 6 is an explanatory diagram showing size information calculated by the brightness analysis processing according to the second embodiment.

FIG. 7 is a flowchart relating to selection of a training endoscopic image according to the second embodiment.

FIG. 8 is an explanatory diagram showing positional information calculated by the brightness analysis processing according to a first modification example.

FIG. 9 is an explanatory diagram showing brightness analysis processing and selection processing according to a third embodiment.

FIG. 10 is an explanatory diagram showing the selection processing according to the third embodiment, which is processing in a case of calculating a ratio of the total number of pixels of a target region to the number of pixels of an endoscopic image.

FIG. 11 is a flowchart relating to selection of a training endoscopic image according to the third embodiment.

FIG. 12 is a block diagram showing a function of a medical image processing device according to a second modification example.

### DESCRIPTION OF THE PREFERRED EMBODIMENTS

### First Embodiment

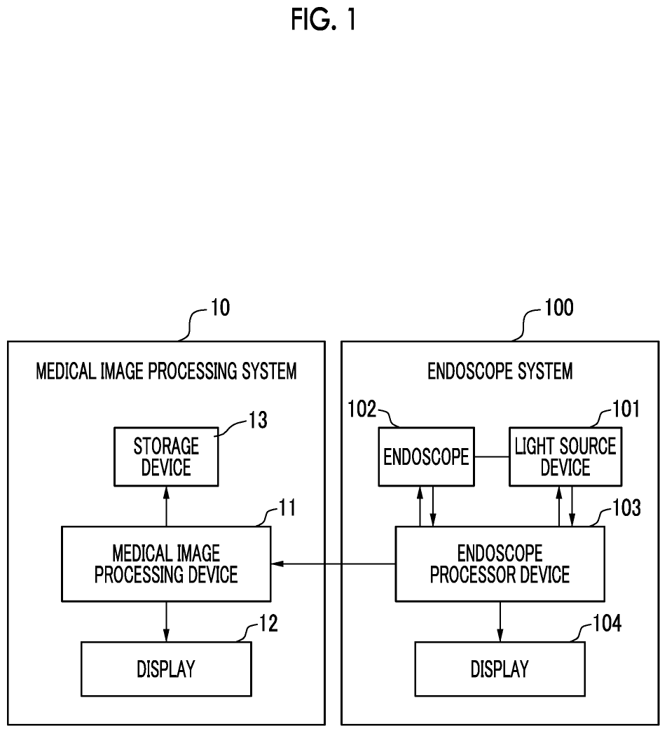

As shown in FIG. 1, a medical image processing system 10 is connected to an endoscope system 100. The endoscope system 100 acquires an endoscopic video image obtained by imaging an inside of a body, such as a digestive tract.

The endoscope system 100 comprises a light source device 101, an endoscope 102, an endoscope processor device 103, and a display 104. The light source device 101 supplies illumination light to be emitted to the subject to the endoscope 102. The endoscope 102 acquires an endoscopic video image by emitting at least one of light in a white wavelength range or light in a specific wavelength range to image the subject. The light in the specific wavelength range that is used as the illumination light by the endoscope 102 is, for example, light in a shorter wavelength range than a green wavelength range, particularly, light in a blue range or a violet range of a visible range.

The endoscope processor device 103 sequentially acquires the endoscopic video image captured by the endoscope 102, and performs various pieces of image processing on the acquired endoscopic video image. The endoscopic video image subjected to various pieces of image processing is displayed on the display 104. The endoscopic video image before or after performing various pieces of image processing is transmitted from the endoscope processor device 103 to the medical image processing system 10.

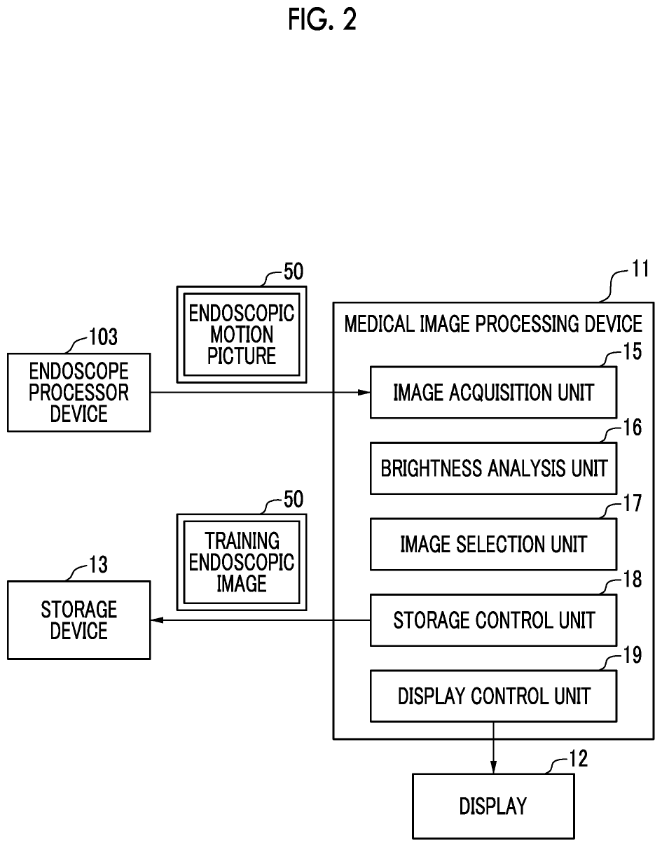

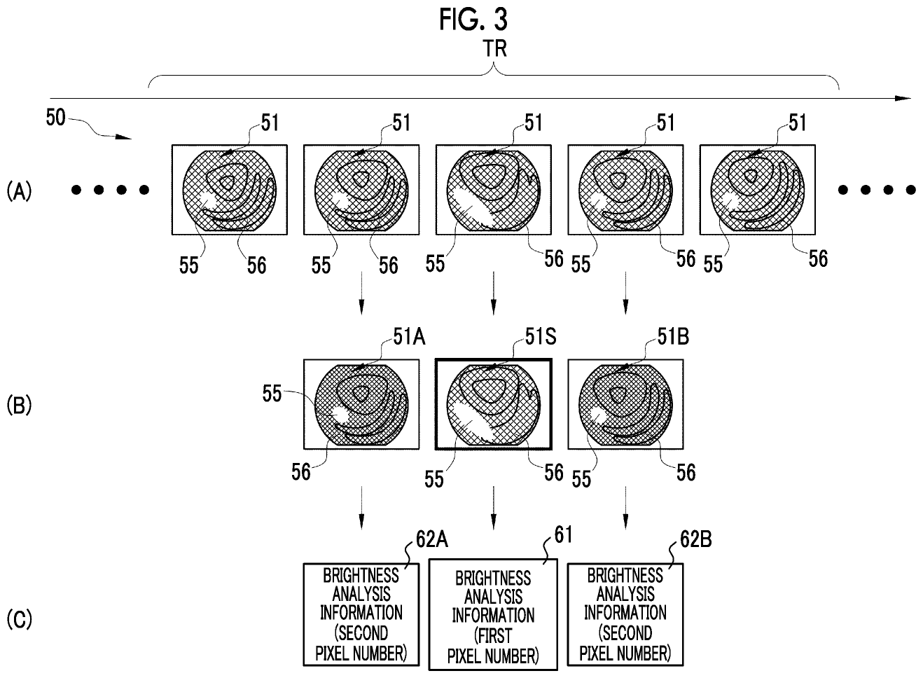

It should be noted that an endoscopic video image 50 (medical video image) (see FIG. 2) transmitted from the endoscope processor device 103 to the medical image processing system 10 is composed of a plurality of endoscopic images 51 (medical images) (see FIG. 3) captured by the endoscope 102 in a time series manner, and a frame image constituting the endoscopic video image 50 can be acquired as the endoscopic image 51 of a still picture after the examination. In addition, the endoscopic video image 50 includes an endoscopic video image captured by a doctor by operating the endoscope 102 or the like, and an endoscopic video image automatically captured without an imaging instruction by the doctor.

The medical image processing system 10 comprises a medical image processing device 11, a display 12, and a storage device 13. It should be noted that the display 12 is provided separately from the display 104 of the endoscope system, the display 12 may be eliminated in the medical image processing system 10 and the display 104 may be used in two uses.

As shown in FIG. 2, the medical image processing device 11 acquires the endoscopic video image 50 transmitted from the endoscope system 100. The medical image processing device 11 comprises an image acquisition unit 15, a brightness analysis unit 16, an image selection unit 17, a storage control unit 18, and a display control unit 19. The image acquisition unit 15 sequentially acquires the endoscopic video image 50 transmitted from the endoscope processor device 103 of the endoscope system 100.

The medical image processing device 11 is composed of a well-known computer, and a program relating to various pieces of processing are incorporated in a program memory (not shown). The medical image processing device 11 is provided with a central control unit (not shown) composed of a processor. By the central control unit executing the program in the program memory, the functions of the image acquisition unit 15, the brightness analysis unit 16, the image selection unit 17, the storage control unit 18, and the display control unit 19 are realized.

The brightness analysis unit 16 analyzes brightness information of each of a plurality of specific endoscopic images (specific medical images) within a specific time range among the plurality of endoscopic images constituting the endoscopic video image 50 to output brightness analysis information. The brightness analysis information output by the brightness analysis unit 16 will be described below.

The image selection unit 17 selects a training endoscopic image 52 (training medical image) to be used for machine learning from among the plurality of specific endoscopic images using the brightness analysis information output by the brightness analysis unit 16. The storage control unit 18 stores the training endoscopic image 52 selected by the image selection unit 17 in the storage device 13. The display control unit 19 causes the display 12 to display a setting screen or the like on which a user performs a setting operation in a case in which the medical image processing device 11 executes various pieces of processing.

The storage device 13 is a hard disk drive which is built in the medical image processing device 11 or connected to the medical image processing device 11 via a cable or the network, or a disk array in which a plurality of hard disk drives are mounted.

Analysis processing in which the brightness analysis unit 16 outputs the brightness analysis information will be described. As shown in (A) of FIG. 3, the brightness analysis unit 16 analyzes the brightness information of each of the plurality of specific endoscopic images (specific medical images) within a specific time range TR among the plurality of endoscopic images 51 constituting the endoscopic video image 50. The specific time range TR may be, for example, an entire time range in a case in which the endoscopic video image 50 is captured, or a time range excluding a certain time after the start of examination and the end of examination from the time range in which the endoscopic video image 50 is captured. It is preferable that the specific time range TR be determined in a time range in which an imaging position of the endoscope is not significantly changed, the specific time range TR is preferably 15 seconds, and more preferably 5 seconds. In addition, for the specific time range TR, the time range may be determined based on a recognition result of an imaging scene of the endoscopic image.

As shown in (B) of FIG. 3, the brightness analysis unit 16 analyzes the brightness information of each of a target endoscopic image 51S (target medical image) and peripheral endoscopic images 51A and 51B (peripheral medical images) different from the target endoscopic image 51S, as the plurality of specific endoscopic images within the specific time range TR. In an example shown in (B) of FIG. 3, the brightness analysis unit 16 uses, as the peripheral endoscopic images 51A and 51B, the endoscopic images 51 which are frame image captured immediately before and immediately after the target endoscopic image 51S among the plurality of endoscopic images 51 arranged in a time series manner.

In an examples shown in (A) and (B) of FIG. 3, a low image quality region having the brightness equal to or greater than a certain threshold value is indicated by a reference numeral 55, and other normal image quality regions are indicated by a reference numeral 56. It should be noted that, in (A) and (B) of FIG. 3, although it is different from the actual endoscopic image 51, the low image quality region 55 is shown as a blank part and the normal image quality region 56 is shown as a shaded part for convenience of illustration. The low image quality region 55 having the brightness equal to or greater than the certain threshold value is often generated in the endoscopic image 51 in a case in which imaging is performed in a state in which halation, blurriness, blur, or water fog occurs, or in a state of being in close contact with a mucous membrane. Therefore, the brightness analysis information of the low image quality region 55 serves as a reference in a case of selecting the training endoscopic image 52. In the present embodiment, a preset value is used for the certain threshold value used in a case of outputting the brightness analysis information.

As shown in (C) of FIG. 3, the brightness analysis unit 16 outputs, as the brightness analysis information, size information of the low image quality region 55 having the brightness equal to or greater than the certain threshold value for each of the target endoscopic image 51S, and the peripheral endoscopic images 51A and 51B. Specifically, the brightness analysis unit 16 calculates a first pixel number 61, which is the number of pixels of the low image quality region 55 having the brightness equal to or greater than the certain threshold value, for the target endoscopic image 51S, and calculates second pixel numbers 62A and 62B, which are the number of pixels of the low image quality region 55 having the brightness equal to or greater than the certain threshold value, for the peripheral endoscopic images 51A and 51B. It should be noted that the first pixel number 61 is obtained by counting the number of pixels included in the low image quality region 55 of the target endoscopic image 51S, and the second pixel numbers 62A and 62B are obtained by counting the number of pixels included in the low image quality regions 55 of the peripheral endoscopic images 51A and 51B, respectively. It should be noted that a region specified by image recognition processing may be used as the low image quality region 55. The image including a low image quality region, such as halation, blurriness, blur, or water fog may be learned in advance, and the low image quality region may be specified by using the trained model.

The brightness analysis unit 16 outputs the first pixel number 61, and the second pixel numbers 62A and 62B as the brightness analysis information. The first pixel number 61, and the second pixel numbers 62A and 62B output from the brightness analysis unit 16 are input to the image selection unit 17 together with the target endoscopic image 51S.

The image selection unit 17 determines whether or not to select the target endoscopic image 51S as the training endoscopic image 52 by comparing the first pixel number 61 with the second pixel numbers 62A and 62B. In the present embodiment, in a case in which the first pixel number 61 is greater than a maximum value of the second pixel numbers 62A and 62B (that is, the largest value in the second pixel numbers 62A and 62B), the image selection unit 17 excludes the target endoscopic image 51S from the training endoscopic image 52.

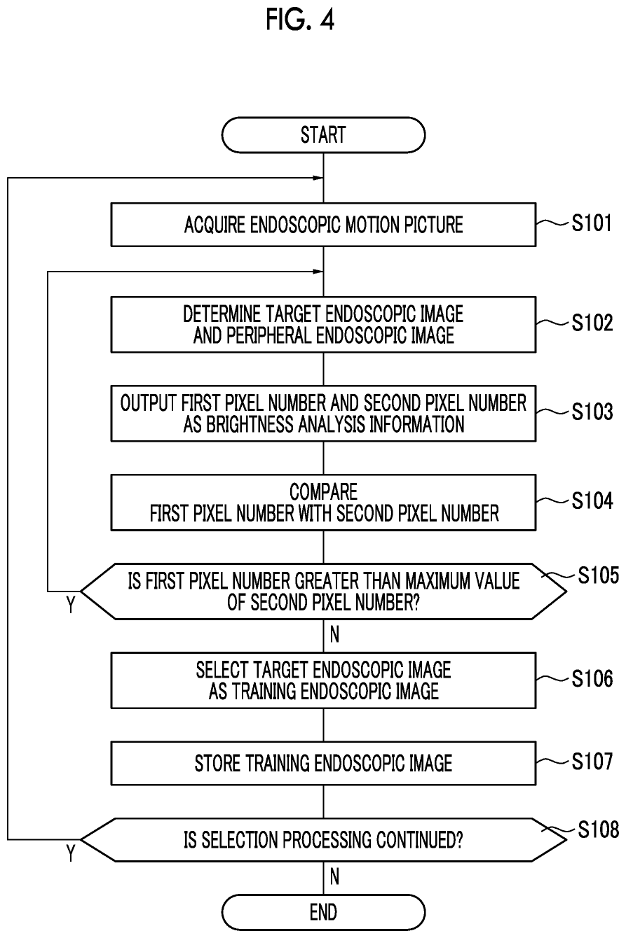

A series of flows in which the medical image processing device 11 acquires the endoscopic video image 50 and selects the training endoscopic image 52 to be used for machine learning will be described with reference to a flowchart shown in FIG. 4. The image acquisition unit 15 sequentially acquires the endoscopic video image 50 from the endoscope processor device 103 (S101). First, the brightness analysis unit 16 determines the target endoscopic image 51S, and the peripheral endoscopic images 51A and 51B from among the plurality of endoscopic images constituting the endoscopic video image 50 (S102). The brightness analysis unit 16 calculates the first pixel number 61 for the target endoscopic image 51S, calculates the second pixel numbers 62A and 62B for the peripheral endoscopic images 51A and 51B, and outputs the calculated pixel numbers as the brightness analysis information (S103).

The image selection unit 17 compares the first pixel number 61 with the second pixel numbers 62A and 62B (S104). In a case in which the first pixel number 61 is greater than the maximum value of the second pixel numbers 62A and 62B (Y in S105), the target endoscopic image 51S is not selected as the training endoscopic image 52, and in a case in which the first pixel number 61 is equal to or less than the maximum value of the second pixel numbers 62A and 62B (N in S105), the target endoscopic image 51S is selected as the training endoscopic image 52 (S106).

The storage control unit 18 stores the target endoscopic image 51S selected by the image selection unit 17 in the storage device 13 as the training endoscopic image 52 (S107). It should be noted that, in a case in which the first pixel number 61 is greater than the maximum value of the second pixel numbers 62A and 62B (Y in S105) and the target endoscopic image 51S is not selected as the training endoscopic image 52, the storage control unit 18 does not store the target endoscopic image 51S in the storage device 13.

Thereafter, the selection processing is continued (Y in S108), the same processing is repeated for the endoscopic image 51 included in the specific time range TR (S101 to S107), and in a case in which the selection processing is performed on all of endoscopic images 51 included in the specific time range TR (N in S108), the medical image processing device 11 ends all the processing.

As described above, since the medical image processing device 11 outputs the brightness analysis information for the specific endoscopic image among the plurality of endoscopic images 51 constituting the endoscopic video image 50 to select the training endoscopic image 52 using the brightness analysis information, the endoscopic image 51 having a low image quality can be automatically excluded. That is, it is possible to efficiently select the endoscopic image suitable for machine learning. Therefore, it is not necessary for the doctor who is the user to visually confirm and exclude the endoscopic image having a low image quality, and the labor or work time of the doctor can be saved.

In addition, since the medical image processing device 11 calculates the first pixel number 61 which is the number of pixels of the low image quality region 55 for the target endoscopic image 51S, calculates the second pixel numbers 62A and 62B which are the number of pixels of the low image quality region 55 for the peripheral endoscopic images 51A and 51B, and selects the training endoscopic image 52 by comparing the first pixel number 61 with the second pixel numbers 62A and 62B, the endoscopic image suitable for machine learning can be further accurately and efficiently selected.

In the first embodiment, the frame images captured immediately before and immediately after the target endoscopic image 51S are used as the peripheral endoscopic images 51A and 51B, respectively, but the number of peripheral endoscopic images is not limited to this, a total of three or more frame images captured before and after the target endoscopic image 51S may be used as the peripheral endoscopic images. In a case in which a plurality of peripheral endoscopic images are used, the size information of a plurality of low image quality regions 55 may be averaged and compared with the target endoscopic image.

In the first embodiment, the first pixel number 61 is calculated for the target endoscopic image 51S, the second pixel numbers 62A and 62B are calculated for the peripheral endoscopic images 51A and 51B, and in a case in which the first pixel number 61 is greater than the maximum value of the second pixel numbers 62A and 62B, the target endoscopic image 51S is excluded from the training endoscopic image 52, but the present invention is not limited to this, the target endoscopic image 51S may be excluded from the training endoscopic image 52 in a case in which the first pixel number 61 is greater than the median value or the average value of the second pixel numbers 62A and 62B.

### Second Embodiment

In the first embodiment, the first pixel number is calculated for the target endoscopic image and the second pixel number is calculated for the peripheral endoscopic image among the plurality of endoscopic images constituting the endoscopic video image, but the present invention is not limited to this. The training endoscopic image 52 need only be selected by outputting, as the brightness analysis information, the size information of the low image quality region having the brightness equal to or greater than the certain threshold value for each of the plurality of specific endoscopic images and comparing the size information of the specific endoscopic images. In the present embodiment, the training endoscopic image 52 is selected by comparing dimensions of the low image quality region as the size information. It should be noted that a medical image processing device to which the present embodiment is applied is the same as the medical image processing device 11 according to the first embodiment except for the brightness analysis and the image selection processing, the same reference numerals are given to the same configurations and functions, and the description thereof will be omitted.

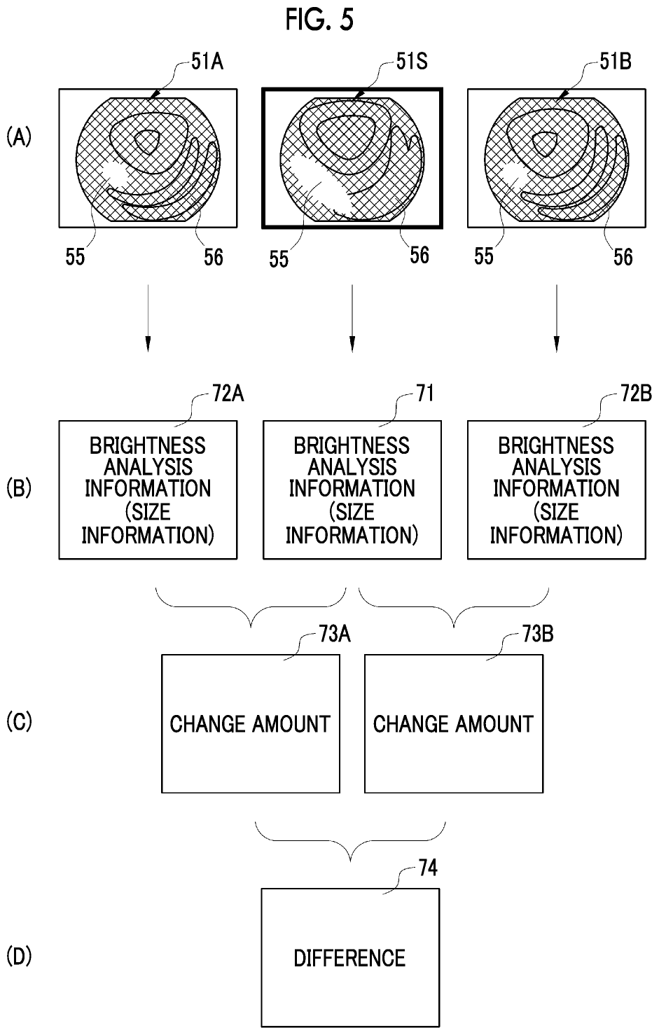

As shown in (A) of FIG. 5, the brightness analysis unit 16 analyzes the brightness information of each of the target endoscopic image 51S (target medical image) and the peripheral endoscopic images 51A and 51B (peripheral medical images) different from the target endoscopic image 51S among the plurality of endoscopic images constituting the endoscopic video image 50, as the plurality of specific endoscopic images within the specific time range TR.

It should be noted that the image acquisition unit 15 sequentially acquires the endoscopic video image 50 transmitted from the endoscope processor device 103 of the endoscope system 100, in the same manner as in the first embodiment. In an example shown in (A) of FIG. 5, the brightness analysis unit 16 uses, as the peripheral endoscopic images 51A and 51B, the endoscopic images 51 which are the frame image captured immediately before and immediately after the target endoscopic image 51S among the plurality of endoscopic images 51 arranged in a time series manner, in the same manner as in the first embodiment.

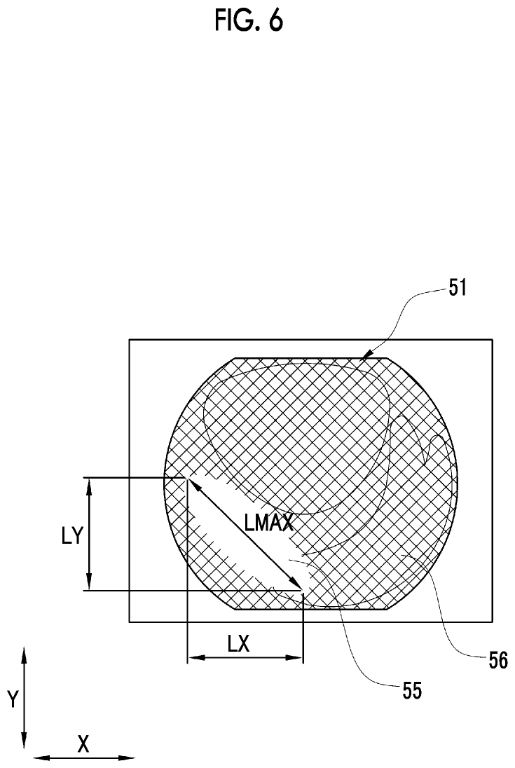

As shown in (B) of FIG. 5, the brightness analysis unit 16 outputs, as size information 71, 72A, and 72B, the dimensions of the low image quality region 55 having the brightness equal to or greater than the certain threshold value for each of the target endoscopic image 51S, and the peripheral endoscopic images 51A and 51B. Specifically, the brightness analysis unit 16 outputs, as the size information 71, 72A, and 72B, a maximum dimension LMAX (see FIG. 6) of the low image quality region 55 having the brightness equal to or greater than the certain threshold value for each of the target endoscopic image 51S, and the peripheral endoscopic images 51A and 51B. It should be noted that the size information output by the brightness analysis unit 16 is not limited to this, and need only be any size information as long as it relates to the dimension of the low image quality region 55 having the brightness equal to or greater than the certain threshold value. For example, a dimension LX (see FIG. 6) in a right-left direction X of the endoscopic image 51, a dimension LY (see FIG. 6) in an up-down direction Y, and the like may be used as the size information.

The image selection unit 17 determines whether or not to select the target endoscopic image 51S as the training endoscopic image 52 by comparing the size information 71, 72A, and 72B of the target endoscopic image 51S, and the peripheral endoscopic images 51A and 51B.

In the present embodiment, as shown in (C) of FIG. 5, the image selection unit 17 calculates a change amount 73A between the size information 72A and the size information 71, and a change amount 73B between the size information 71 and the size information 72B. The image selection unit 17 determines whether or not to select the target endoscopic image 51S as the training endoscopic image 52 using the change amounts 73A and 73B. For example, in a case in which any of the change amount 73A or the change amount 73B is greater than the certain threshold value, the target endoscopic image 51S is excluded from the training endoscopic image 52.

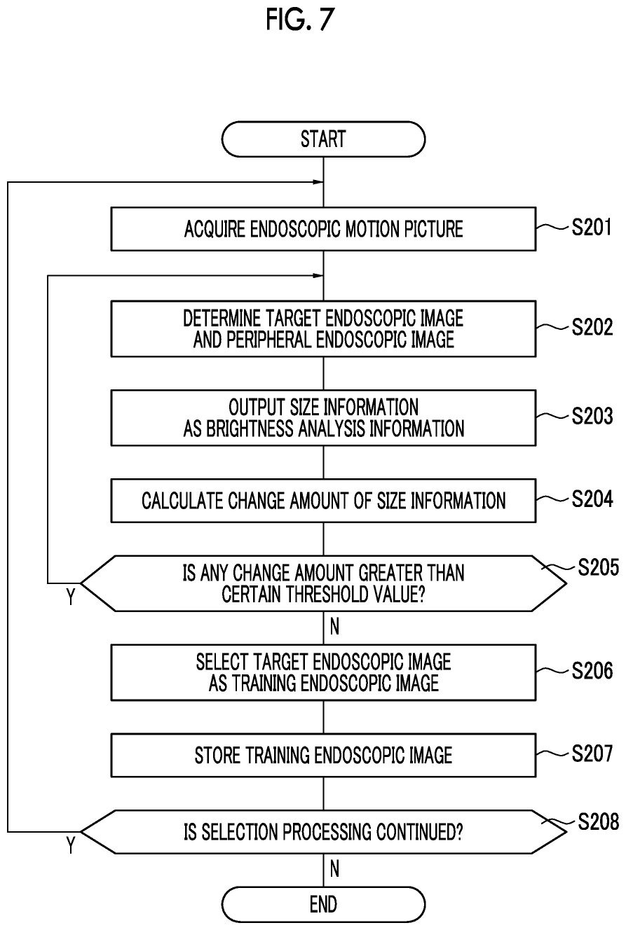

A series of flows in which the medical image processing device according to the present embodiment selects the training endoscopic image 52 to be used for machine learning will be described with reference to a flowchart shown in FIG. 7. Processing of acquiring the endoscopic video image 50 (S201) and processing of determining the target endoscopic image 51S, and the peripheral endoscopic images 51A and 51B (S202) are the same as in S101 and S102 of the flowchart in the first embodiment. The brightness analysis unit 16 outputs, as the brightness analysis information, the size information 71, 72A, and 72B which are the dimensions of the low image quality region 55 having the brightness equal to or greater than the certain threshold value for the target endoscopic image 51S, and the peripheral endoscopic images 51A and 51B (S203).

As described above, the image selection unit 17 calculates the change amount 73A between the size information 72A and the size information 71, and the change amount 73B between the size information 71 and the size information 72B (S204). In a case in which any of the change amount 73A or the change amount 73B is greater than the certain threshold value (Y in S205), the target endoscopic image 51S is not selected as the training endoscopic image 52. On the other hand, in a case in which both the change amounts 73A and 73B are less than the certain threshold value (N in S205), the image selection unit 17 selects the target endoscopic image 51S as the training endoscopic image 52 (S206). The storage control unit 18 stores the target endoscopic image 51S selected by the image selection unit 17 in the storage device 13 as the training endoscopic image 52 (S207). It should be noted that, in a case in which the target endoscopic image 51S is not selected as the training endoscopic image 52 (Y in S205), the storage control unit 18 does not store the target endoscopic image 51S in the storage device 13.

Thereafter, the selection processing is continued (Y in S208), the same processing is repeated for the endoscopic image 51 included in the specific time range TR (S201 to S207), and in a case in which the selection processing is performed on all of endoscopic images 51 included in the specific time range TR (N in S208), the medical image processing device 11 ends all the processing.

As described above, in the medical image processing device according to the present embodiment, the size information is output, as the brightness analysis information, for the target endoscopic image 51S, and the peripheral endoscopic images 51A and 51B among the plurality of endoscopic images 51 constituting the endoscopic video image 50 to select the training endoscopic image 52 using the size information. Therefore, in the same manner as in the first embodiment, it is possible to efficiently select the endoscopic image suitable for machine learning, and it is possible to save the labor or work time of the doctor.

In the second embodiment, the image selection unit 17 determines whether or not to select the target endoscopic image 51S as the training endoscopic image 52 by using the change amount 73A between the size information 72A and the size information 71, and the change amount 73B between the size information 71 and the size information 72B, but the present invention is not limited to this, and the image selection unit 17 may determine whether or not to select the target endoscopic image 51S as the training endoscopic image 52 by using a difference 74 (see (D) of FIG. 5) between the change amount 73A and the change amount 73B. In this case, in a case in which the difference 74 is greater than the certain threshold value, the target endoscopic image 51S is excluded from the training endoscopic image 52.

### First Modification Example

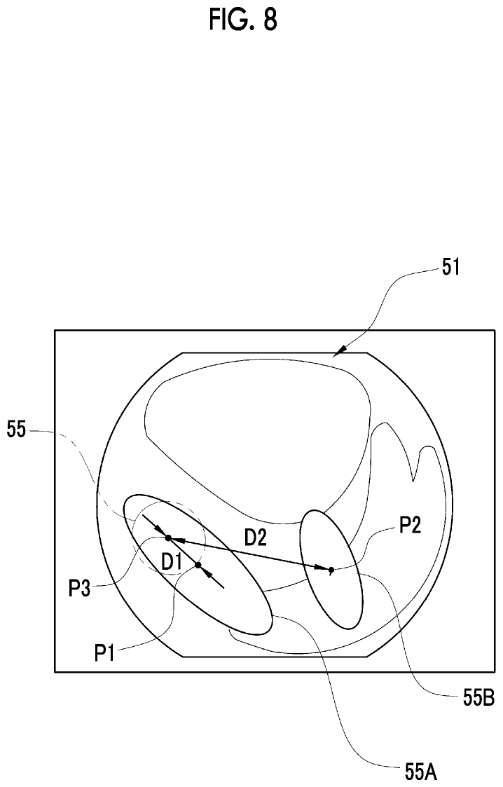

In the second embodiment, the image selection unit 17 selects the training endoscopic image 52 by using the size information of the target endoscopic image 51S, and the peripheral endoscopic images 51A and 51B, but the present invention is not limited to this. By outputting, as the brightness analysis information, the size information and the positional information of the low image quality region 55 having the brightness equal to or greater than the certain threshold value for each of the plurality of specific endoscopic images, the image selection unit 17 may compare the size information and the positional information of the plurality of specific endoscopic images to select the training endoscopic image 52. That is, in addition to the comparison of the size information of the low image quality region 55 described in the first and second embodiments, the positional information of the low image quality region 55 is compared as shown in FIG. 8.

In FIG. 8, low image quality regions 55A and 55B of the target endoscopic images 51S are shown by solid lines, and the low image quality regions 55 of the peripheral endoscopic images 51A and 51B are shown by two-dot chain lines. As positional information P1 to P3 of the low image quality region 55, for example, the coordinates of the centroid of the low image quality region 55 are calculated.

For example, in a case in which there are the plurality of low image quality regions 55 of the target endoscopic image 51S (two in an example shown in FIG. 8), distances D1 and D2 between the positional information P1 and P2 of the low image quality regions 55A and 55B in the target endoscopic image 51S, and the positional information P3 of the low image quality regions 55C of the peripheral endoscopic images 51A and 51B are calculated. Then, the image selection unit 17 uses, as a comparison target, the low image quality region 55A close to the positional information P3 of the low image quality regions 55 of the peripheral endoscopic images 51A and 51B, that is, having short distances D1 and D2. Specifically, the image selection unit 17 associates the low image quality regions 55 of the peripheral endoscopic images 51A and 51B with the low image quality region 55A in the target endoscopic image 51S. Next, the image selection unit 17 selects the training endoscopic image 52 by using the size information of the low image quality region 55A and the size information of the low image quality region 55A and the size information of the low image quality regions 55 of the peripheral endoscopic images 51A and 51B, in the same manner as in each of the embodiments.

### Third Embodiment

In the first and second embodiments, the training endoscopic image 52 is selected by outputting the size information for each of the target endoscopic image and the peripheral endoscopic image, and comparing the size information of the target endoscopic image and the peripheral endoscopic image. However, in the present embodiment, the training endoscopic image 52 is selected by dividing the target endoscopic image into a plurality of regions, outputting divided region brightness analysis information for each of divided regions of the target endoscopic image, calculating peripheral brightness analysis information for the peripheral endoscopic image, and comparing the divided region brightness analysis information with the peripheral brightness analysis information. It should be noted that a medical image processing device to which the present embodiment is applied is the same as the medical image processing device 11 according to the first embodiment except for the processing of the brightness analysis and the image selection, the same reference numerals are given to the same configurations and functions, and the description thereof will be omitted.

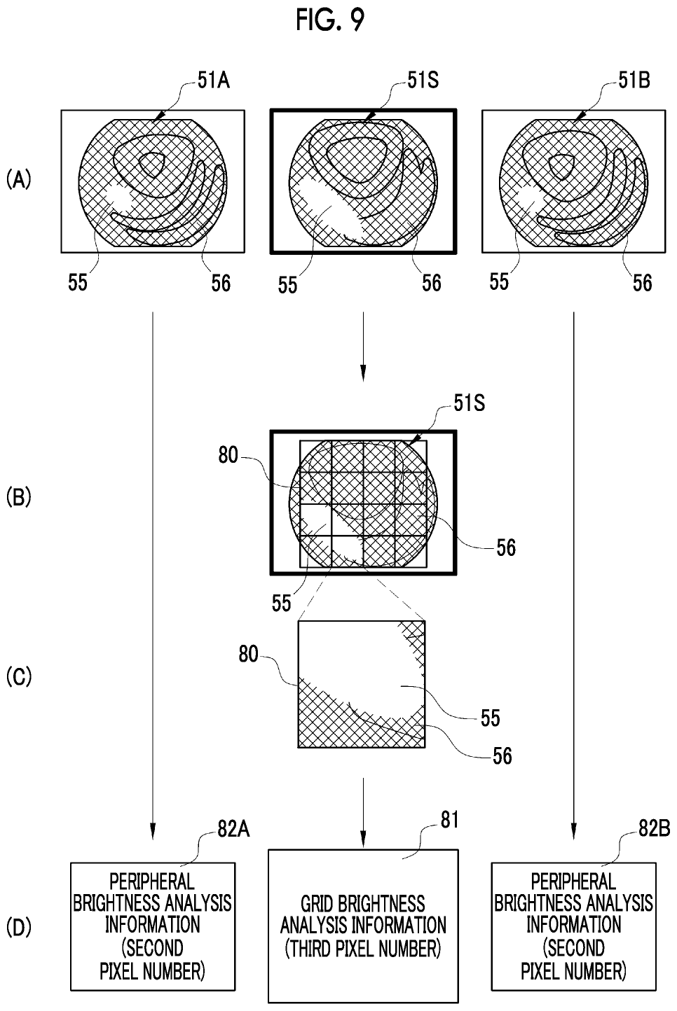

As shown in (A) of FIG. 9, the brightness analysis unit 16 analyzes the brightness information of each of the target endoscopic image 51S (target medical image) and the peripheral endoscopic images 51A and 51B (peripheral medical images) different from the target endoscopic image 51S among the plurality of endoscopic images constituting the endoscopic video image 50, as the plurality of specific endoscopic images within the specific time range TR.

It should be noted that the image acquisition unit 15 sequentially acquires the endoscopic video image 50 transmitted from the endoscope processor device 103 of the endoscope system 100, in the same manner as in the first embodiment. In an example shown in (A) of FIG. 9, the brightness analysis unit 16 uses, as the peripheral endoscopic images 51A and 51B, the endoscopic images 51 which are the frame image captured immediately before and immediately after the target endoscopic image 51S among the plurality of endoscopic images 51 arranged in a time series manner, in the same manner as in the first embodiment.

As shown in (B) of FIG. 9, the brightness analysis unit 16 divides the target endoscopic image 51S into a plurality of grids 80 (plurality of regions). The grid 80 refers to each square region obtained by dividing the target endoscopic image 51S into squares. In an example shown in (B) of FIG. 9, the target endoscopic image 51S is divided into 4×4 squares, and a total of 16 grids 80 are set. It should be noted that the size and number of divisions of the grid 80 are not limited to this, and may be appropriately changed depending on the number of pixels of the endoscopic image 51, the processing capacity of the brightness analysis unit 16, and the like. It should be noted that (C) of FIG. 9 is an enlarged view of one of the grids 80 obtained by dividing the target endoscopic image 51S with respect to (B) of FIG. 9.

As shown in (D) of FIG. 9, the brightness analysis unit 16 calculates grid brightness analysis information (divided region brightness analysis unit) of the low image quality region 55 having the brightness equal to or greater than the certain threshold value for each of the grids 80 of the target endoscopic image 51S. In the present embodiment, the brightness analysis unit 16 calculates a third pixel number 81, which is the number of pixels of the low image quality region 55, as the grid brightness analysis information for each of the grids 80. It should be noted that the third pixel number 81 is obtained by counting the number of pixels included in the low image quality region 55 for each of the grids 80.

On the other hand, as shown in (D) of FIG. 9, the brightness analysis unit 16 calculates, as the peripheral brightness analysis information, second pixel numbers 82A and 82B, which are the number of pixels of the low image quality region 55 having the brightness equal to or greater than the certain threshold value, for the peripheral endoscopic images 51A and 51B. It should be noted that the second pixel numbers 82A and 82B calculated by the brightness analysis unit 16 are the same as the second pixel numbers 62A and 62B calculated by the brightness analysis unit 16 in the first embodiment.

The brightness analysis unit 16 outputs the third pixel number 81 and the second pixel numbers 82A and 82B as the brightness analysis information. The third pixel number 81 and the second pixel numbers 82A and 82B output from the brightness analysis unit 16 are input to the image selection unit 17 together with the target endoscopic image 51S.

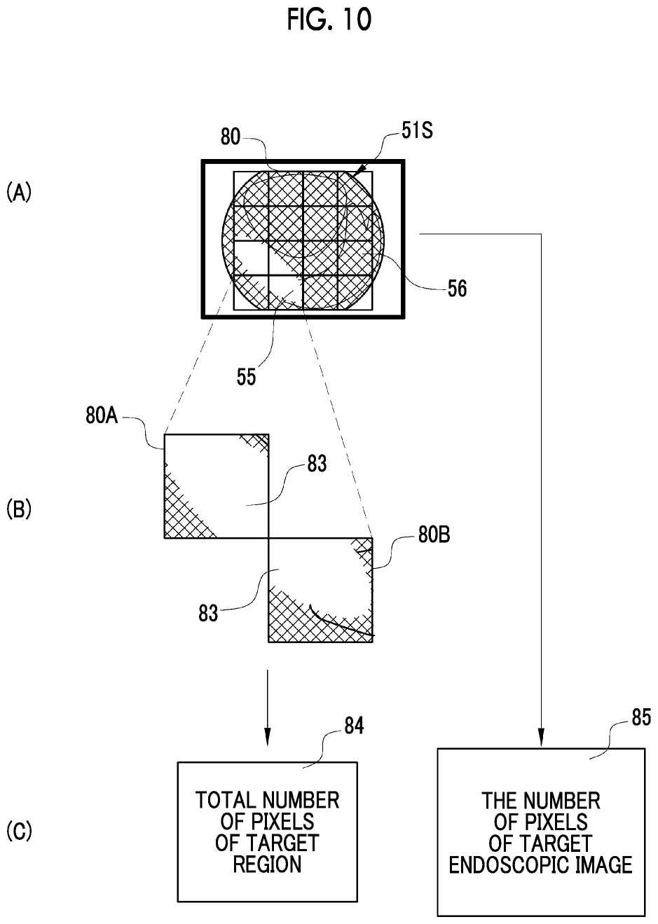

The image selection unit 17 extracts the low image quality region 55 of the grid 80 as the target region 83 by comparing the second pixel numbers 82A and 82B of the peripheral endoscopic images 51A and 51B with the third pixel number 81 of at least one grid 80 (see (B) of FIG. 10). Specifically, in a case in which the third pixel number 81 of the grid 80 is greater than the maximum value of the second pixel numbers 82A and 82B of the peripheral endoscopic images 51A and 51B, the low image quality region 55 of the grid 80 corresponding to the third pixel number 81 is used as the target region 83. Then, in a case in which a total number of pixels 84 (see (C) of FIG. 10) of the target region 83 is equal to or greater than a certain ratio of the number of pixels 85 of the target endoscopic image 51S (see (C) of FIG. 10), the image selection unit 17 excludes the target endoscopic image 51S from the training endoscopic image 52. It should be noted that the present invention is not limited to this, and the image selection unit 17 may exclude the target endoscopic image 51S from the training endoscopic image 52 in a case in which the maximum value, the average value, or the median value of the number of pixels of the target region 83 is equal to or greater than the certain ratio of the number of pixels of the target endoscopic image 51S.

In an example shown in FIG. 10, among the plurality of grids 80 (see (A) of FIG. 10) obtained by dividing the target endoscopic image 51S, the third pixel number 81 of the two grids 80A and 80B is greater than the maximum value of the second pixel numbers 82A and 82B of the peripheral endoscopic images 51A and 51B. Therefore, the image selection unit 17 uses the low image quality regions 55 of the grids 80A and 80B corresponding to the third pixel number 81 as the target region 83 (see (B) of FIG. 10). It should be noted that (B) of FIG. 10 is an enlarged view of the grids 80A and 80B including the target region 83 with respect to (A) of FIG. 10.

Then, in a case in which the total number of pixels 84 of the target region 83, that is the total number of pixels 84 of the target region 83 in the grids 80A and 80B is equal to or greater than the certain ratio of the number of pixels 85 of the target endoscopic image 51S, the image selection unit 17 excludes the target endoscopic image 51S from the training endoscopic image 52.

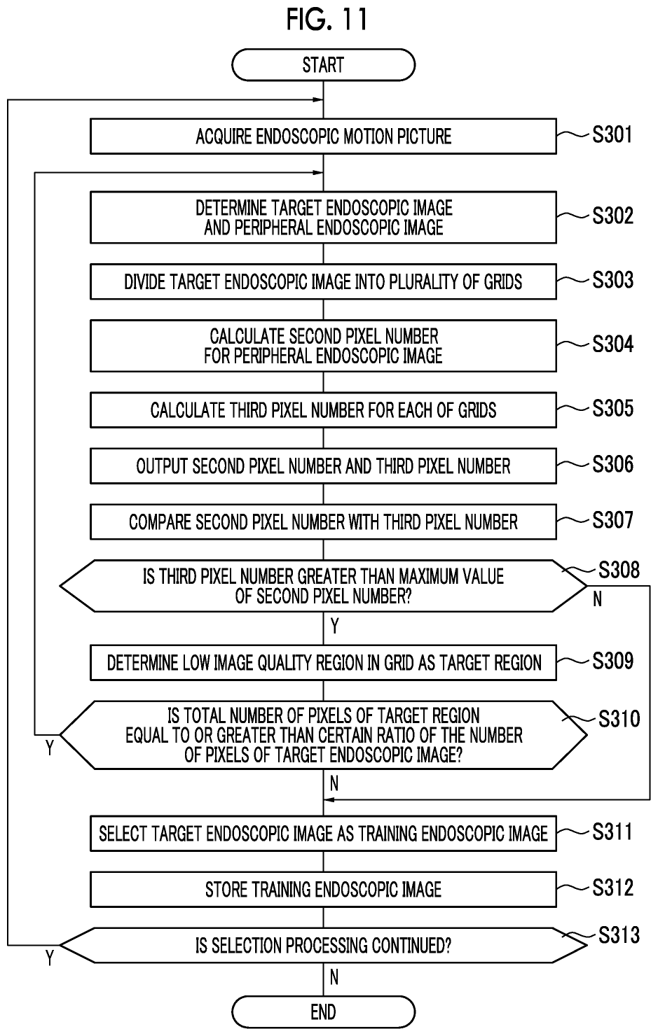

A series of flows in which the medical image processing device according to the present embodiment selects the training endoscopic image 52 to be used for machine learning will be described with reference to a flowchart shown in FIG. 11. Processing of acquiring the endoscopic video image 50 (S301) and processing of determining the target endoscopic image 51S, and the peripheral endoscopic images 51A and 51B (S302) are the same as in S101 and S102 of the flowchart in the first embodiment. The brightness analysis unit 16 divides the target endoscopic image 51S into the plurality of grids 80 (S303).

The brightness analysis unit 16 calculates the second pixel numbers 82A and 82B, which are the number of pixels of the low image quality region 55 having the brightness equal to or greater than the certain threshold value, for the peripheral endoscopic images 51A and 51B, respectively (S304). On the other hand, the brightness analysis unit 16 calculates the third pixel number 81, which is the number of pixels of the low image quality region 55 having the brightness equal to or greater than the certain threshold value, for each of the grids 80 of the target endoscopic image 51S (S305). The second pixel numbers 82A and 82B and the third pixel number 81 are output as the brightness analysis information (S306).

The image selection unit 17 compares the second pixel numbers 82A and 82B of the peripheral endoscopic images 51A and 51B with the third pixel number 81 of at least one grid 80 (S307), and in a case in which the third pixel number 81 is greater than the maximum value of the second pixel numbers 82A and 82B (Y in S308), the image selection unit 17 determines the low image quality region 55 of the grid 80 corresponding to the third pixel number 81 as the target region 83 (S309). It should be noted that, in a case in which the third pixel number 81 is equal to or less than the maximum value of the second pixel numbers 82A and 82B (N in S308), the low image quality region 55 of the grid 80 corresponding to the third pixel number 81 is not used as the target region 83, and the target endoscopic image 51S is selected as the training endoscopic image 52 (S311).

Then, in a case in which the total number of pixels of the target region 83 is equal to or greater than the certain ratio of the number of pixels of the target endoscopic image 51S (Y in S310), the image selection unit 17 does not select the target endoscopic image 51S as the training endoscopic image, and in a case in which the total number of pixels of the target region 83 is less than the certain ratio of the number of pixels of the target endoscopic image 51S (N in S310), the image selection unit 17 selects the target endoscopic image MS as the training endoscopic image 52 (S311).

The storage control unit 18 stores the target endoscopic image 51S selected by the image selection unit 17 in the storage device 13 as the training endoscopic image (S312). It should be noted that, in a case in which the total number of pixels of the target region 83 is less than the certain ratio of the number of pixels of the target endoscopic image 51S (N in S310) and the target endoscopic image 51S is not selected as the training endoscopic image, the storage control unit 18 does not store the target endoscopic image 51S in the storage device 13.

Thereafter, the selection processing is continued (Y in S313), the same processing is repeated for the endoscopic image 51 included in the specific time range TR (S301 to S312), and in a case in which the selection processing is performed on all of endoscopic images 51 included in the specific time range TR (N in S313), the medical image processing device 11 ends all the processing.

As described above, in the medical image processing device of the present embodiment, the target endoscopic image 51S is divided into the plurality of grids 80, the grid brightness analysis information is output for each of the grids 80, the peripheral brightness analysis information is calculated for the peripheral endoscopic images 51A and 51B, and the grid brightness analysis information and the peripheral brightness analysis information are compared to select the training endoscopic image 52. Therefore, in the same manner as in the first embodiment, it is possible to efficiently select the endoscopic image suitable for machine learning, and it is possible to save the labor or work time of the doctor.

In the third embodiment, in a case of determining whether or not to select the target endoscopic image 51S as the training endoscopic image 52, the target endoscopic image 51S is divided into the plurality of grids 80, the third pixel number 81 is output for each of the grids 80, the second pixel numbers 82A and 82B are calculated for the peripheral endoscopic images 51A and 51B, and the target endoscopic image 51S is excluded from the training endoscopic image 52 in a case in which the third pixel number 81 is greater than the maximum value of the second pixel numbers 82A and 82B, but the present invention is not limited to this. The target endoscopic image 51S may be excluded from the training endoscopic image 52 in a case in which the third pixel number is greater than the median value or the average value of the second pixel number.

In the third embodiment, the brightness analysis unit 16 calculates the third pixel number 81, which is the number of pixels of the low image quality region 55, for each of the grids 80 of the target endoscopic image 51S and calculates the second pixel numbers 82A and 82B, which are the number of pixels of the low image quality region 55, for the peripheral endoscopic images 51A and 51B, respectively, and the image selection unit 17 selects the training endoscopic image 52 by using the second pixel numbers 82A and 82B, and the third pixel number 81. However, the size information used for selecting the training endoscopic image 52 is not limited to the second pixel numbers 82A and 82B, and the third pixel number 81, and the size information, such as the dimension of the low image quality region 55, may be used similar to the brightness analysis information in the second embodiment. In this case, the brightness analysis unit 16 may output divided region analysis information, which is the size information of the low image quality region 55, as the brightness analysis information for each of the grids 80 of the target endoscopic image 51S, and may calculate the peripheral brightness analysis information, which is the size information of the low image quality region 55, for the peripheral endoscopic images 51A and 51B, and the image selection unit 17 may compare the grid brightness analysis information with the peripheral brightness analysis information to select the training endoscopic image 52 to be used for machine learning. In this case, for example, it is preferable that the grid brightness analysis information (size information) and the peripheral brightness analysis information (size information) be compared and the target endoscopic image 51S be excluded from the training endoscopic image 52 in a case in which the grid brightness analysis information is greater than the maximum value, the average value, or the median value of the peripheral brightness analysis information.

In addition, in the third embodiment, the image selection unit 17 selects the training endoscopic image 52 by using the size information for each of the grid 80 of the target endoscopic image 51S, and the peripheral endoscopic images 51A and 51B, but the present invention is not limited to this. The size information and the positional information of the low image quality region 55 having the brightness equal to or greater than the certain threshold value may be output as the brightness analysis information for each of the grid 80 of the target endoscopic image 51S, and the peripheral endoscopic images 51A and 51B, the image selection unit 17 may compare the size information and the positional information of the low image quality region 55 for the grid 80 of the target endoscopic image 51S, and the peripheral endoscopic images 51A and 51B to select the training endoscopic image 52 to be used for machine learning from among the plurality of specific medical images. That is, it is preferable to calculate the positional information in the same manner as in the first modification example and compare the positional information of the low image quality region 55, in addition to the comparison of the size information described in the first to third embodiments.

### Second Modification Example

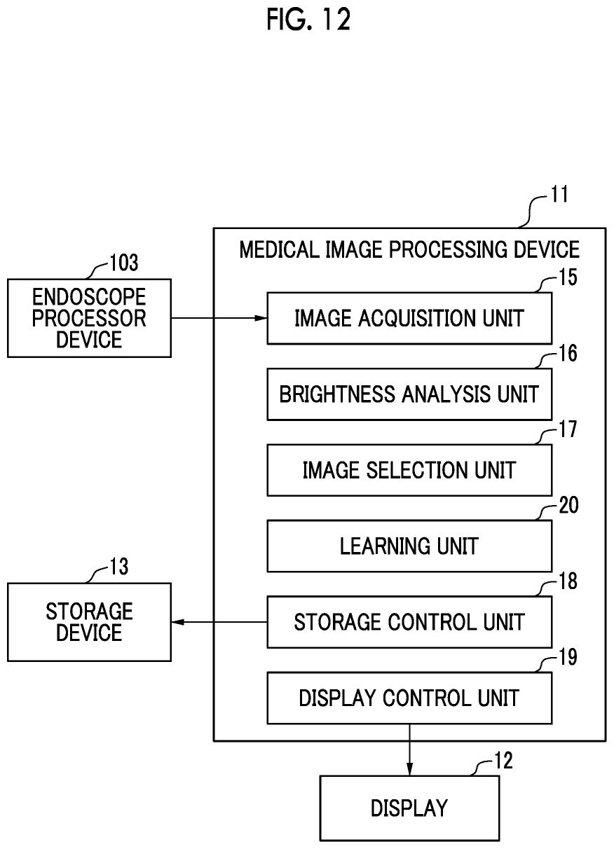

In each of the embodiments described above, the medical image processing device having the functions of the image acquisition unit 15, the brightness analysis unit 16, the image selection unit 17, the storage control unit 18, and the display control unit 19 has been described, but the present invention is not limited to this, and as shown in FIG. 12, a learning unit 20 that performs machine learning may be provided in addition to the configuration described above. In this case, it is preferable that the learning unit 20 perform machine learning on the training endoscopic image 52 selected as in each of the embodiments described above, automatically detect a region of interest, such as a lesion, and detect generate CAD that performs highlighting of the detected region of interest. In addition, machine learning may be performed using the training endoscopic image 52 selected by the image selection unit 17, and may be stored in the storage device 13.

In each of the embodiments described above, the storage control unit 18 stores the training endoscopic image 52 selected by the image selection unit 17 in the storage device 13, and does not store the target endoscopic image 51S that is not selected, but the present invention is not limited to this, and the storage control unit 18 may store the training endoscopic image 52 in a first storage device, and may store the target endoscopic image 51S that is not selected in a second storage device different from the first storage device. In addition, in this case, the training medical image stored in the first storage device may be automatically transmitted to a server or the like provided outside the medical image processing device.

In each of the embodiments described above, the brightness analysis unit 16 uses the preset value for the certain threshold value used in a case of outputting the brightness analysis information, but the present invention is not limited to this, and the brightness analysis unit 16 may record operation information, an imaging condition, or information on an imaging apparatus in a case in which the endoscope system 100 captures the endoscopic image to determine the certain threshold value in accordance with the above information. Specifically, in a case in which the information generating the change of the brightness is recorded, such as a case in which the endoscope 102 is bent and operated (a case in which the inclination of a distal end portion is changed), a case in which a zoom operation is performed, or a case in which the amount of illumination light is changed, it is preferable to change the certain threshold value. In addition, it is preferable to record the operation information, the imaging condition, the information on the imaging apparatus, or the like in association with the endoscopic video image.

In addition, as in the first modification example, the positional information of the low image quality region 55 having the brightness equal to or greater than the certain threshold value may be calculated to determine the certain threshold value in accordance with the positional information. For example, since a lens of the endoscope looks like a fisheye, the size of the low image quality region 55 is larger at an edge of a screen than at the center of the screen, so that the certain threshold value is increased for the low image quality region 55 determined to be the edge of the screen from the positional information. Alternatively, in a case in which it is assumed that a region in which the mucous membrane is oblique to the light source and a front region are mixed in one endoscopic image 51, the size of the low image quality region 55 is larger in the oblique region, so that the certain threshold value is increased for the low image quality region 55 determined to be the region in which the light is obliquely shined from the positional information.

In the endoscope system 100, a capsule endoscope may be used as the endoscope 102. In this case, the light source device 101 and a part of the endoscope processor device 103 can be mounted on the capsule endoscope. In addition, the medical image is not limited to the endoscopic image. For the medical image, such as an X-ray image, a computed tomography (CT) image, and a magnetic resonance (MR) image, the medical video image can be acquired to select the training medical image in the same manner as in each of the embodiments described above.

In each of the embodiments described above, the size information of the region having the brightness equal to or greater than the certain threshold value, that is, the high-brightness (bright) region is compared to exclude the region having the large size information from the training medical image, but the present invention is not limited to this. The size information of the region having the brightness less than the certain threshold value, that is the low-brightness (dark) region may be compared to exclude the region having the large size information from the training medical image.

In each of the embodiments and the modification examples described above, the hardware structure of processing units that execute various pieces of processing, such as the image acquisition unit 15, the brightness analysis unit 16, the image selection unit 17, the storage control unit 18, and the display control unit 19, is various processors as described below. The various processors include a central processing unit (CPU) as a general-purpose processor functioning as various processing units by executing software (program), a programmable logic device (PLD) as a processor of which a circuit configuration can be changed after manufacturing, such as a field programmable gate array (FPGA), a dedicated electric circuit as a processor having a circuit configuration specially designed for executing various pieces of processing, a graphical processing unit (GPU) that performs a large amount of processing, such as image processing, in parallel, and the like.

One processing unit may be composed of one of these various processors, or may be composed of a combination of two or more same or different types of processors (for example, a plurality of FPGAs, a combination of a CPU and an FPGA, or a combination of a CPU and a GPU). In addition, a plurality of the processing units may be composed of one processor. As an example in which the plurality of processing units are composed of one processor, first, there is a form in which one processor is composed of a combination of one or more CPUs and software as represented by a computer, such as a client or a server, and this processor functions as the plurality of processing units. Second, as represented by a system on chip (SoC) or the like, there is a form in which a processor that realizes the function of the entire system including the plurality of processing units with one integrated circuit (IC) chip is used. In this way, various processing units are composed of one or more of the various processors described above as the hardware structure.

More specifically, the hardware structure of these various processors is an electrical circuit (circuitry) in a form in which circuit elements, such as semiconductor elements, are combined.

### EXPLANATION OF REFERENCES

10: medical image processing system
11: medical image processing device
12: display
13: storage device
15: image acquisition unit
16: brightness analysis unit
17: image selection unit
18: storage control unit
19: display control unit
20: learning unit
50: endoscopic video image
51: endoscopic image
51A, 51B: peripheral endoscopic image
51S: target endoscopic image
52: training endoscopic image
55, 55A, 55B: low image quality region
56: normal image quality region
61: first pixel number
62A, 62B: second pixel number
71, 72A, 72B: size information
73A, 73B: change amount
74: difference
80: grid
80A, 80B: grid
81: third pixel number
82A, 82B: second pixel number
83: target region
84: total number of pixels of target region
85: number of pixels of target endoscopic image
100: endoscope system
101: light source device
102: endoscope
103: endoscope processor device
104: display
P1, P2, P3: positional information
LMAX: maximum dimension
LX: dimension in right-left direction
LY: dimension in up-down direction
TR: specific time range
X: right-left direction
Y: up-down direction

What is claimed is:

1. A medical image processing device comprising:
a processor configured to:
acquire a medical video image;
analyze brightness information of each of a plurality of specific medical images within a specific time range among a plurality of medical images constituting the medical video image to output brightness analysis information; and
select a training medical image to be used for machine learning from among the plurality of specific medical images using the brightness analysis information.

2. The medical image processing device according to claim 1,
wherein the processor is configured to:
output, as the brightness analysis information, size information of a region having brightness equal to or greater than a certain threshold value for each of the plurality of specific medical images; and
compare the size information of the plurality of specific medical images to select the training medical image to be used for machine learning from among the plurality of specific medical images.

3. The medical image processing device according to claim 1,
wherein the processor is configured to:
output, as the brightness analysis information, size information and positional information of a region having brightness equal to or greater than a certain threshold value for each of the plurality of specific medical images; and
compare the size information and the positional information of the plurality of specific medical images to select the training medical image to be used for machine learning from among the plurality of specific medical images.

4. The medical image processing device according to claim 2,
wherein the processor is configured to:
calculate the size information for a target medical image and a peripheral medical image different from the target medical image among the plurality of medical images constituting the medical video image;
calculate a change amount of the size information between the target medical image and the peripheral medical image; and
determine whether or not to select the target medical image as the training medical image using the change amount.

5. The medical image processing device according to claim 2,
wherein the processor is configured to:
calculate the size information for a target medical image and a plurality of peripheral medical images different from the target medical image among the plurality of medical images constituting the medical video image;
calculate a difference of a change amount using the size information of the target medical image and the plurality of peripheral medical images; and
determine whether or not to select the target medical image as the training medical image using the difference.

6. The medical image processing device according to claim 1,
wherein the processor is configured to:
calculate a first pixel number which is the number of pixels of the region having the brightness equal to or greater than the certain threshold value for a target medical image among the plurality of specific medical images;
calculate a second pixel number which is the number of pixels of the region having the brightness equal to or greater than the certain threshold value for a peripheral medical image different from the target medical image among the plurality of specific medical images;
output the first pixel number and the second pixel number as the brightness analysis information; and
determine whether or not to select the target medical image as the training medical image by comparing the first pixel number with the second pixel number.

7. The medical image processing device according to claim 6,
wherein the processor is configured to exclude the target medical image from the training medical image in a case in which the first pixel number is greater than a maximum value, an average value, or a median value of the second pixel number.

8. The medical image processing device according to claim 1,
wherein the processor is configured to:
divide a target medical image among the plurality of specific medical images into a plurality of regions;
output, as the brightness analysis information, divided region brightness analysis information which is size information of a region having brightness equal to or greater than a certain threshold value for each of the plurality of regions of the target medical image;
calculate peripheral brightness analysis information which is size information of the region having the brightness equal to or greater than the certain threshold value for a peripheral medical image different from the target medical image; and
compare the divided region brightness analysis information with the peripheral brightness analysis information to select the training medical image to be used for machine learning from among the plurality of specific medical images.

9. The medical image processing device according to claim 1,
wherein the processor is configured to:
divide a target medical image among the plurality of specific medical images into a plurality of regions;
output, as the brightness analysis information, divided region brightness analysis information which is size information and positional information of a region having brightness equal to or greater than a certain threshold value for each of the plurality of regions of the target medical image;
calculate peripheral brightness analysis information which is size information and positional information of the region having the brightness equal to or greater than the certain threshold value for a peripheral medical image different from the target medical image; and
compare the divided region brightness analysis information with the peripheral brightness analysis information to select the training medical image to be used for machine learning from among the plurality of specific medical images.

10. The medical image processing device according to claim 1,
wherein the processor is configured to:
divide a target medical image among the plurality of specific medical images into a plurality of regions;
calculate a second pixel number which is the number of pixels of a region having brightness equal to or greater than a certain threshold value for a peripheral medical image different from the target medical image among the plurality of specific medical images;
calculate a third pixel number which is the number of pixels of the region having the brightness equal to or greater than the certain threshold value for each of the plurality of regions;
output the second pixel number and the third pixel number as the brightness analysis information;
extract, as a target region, the region having the brightness equal to or greater than the certain threshold value among the plurality of regions by comparing the second pixel number with the third pixel number in at least one of the plurality of regions; and
determine whether or not to select the target medical image as the training medical image by comparing the target medical image with the target region.

11. The medical image processing device according to claim 10,
wherein the processor is configured to use, as the target region, the region having the brightness equal to or greater than the certain threshold value in the plurality of regions corresponding to the third pixel number in a case in which the third pixel number is greater than a maximum value, an average value, or a median value of the second pixel number.

12. The medical image processing device according to claim 10,
wherein the processor is configured to exclude the target medical image from the training medical image in a case in which a total number of pixels of the target region is equal to or greater than a certain ratio of the number of pixels of the target medical image.

13. The medical image processing device according to claim 10,
wherein the processor is configured to exclude the target medical image from the training medical image in a case in which a maximum value, an average value, or a median value of the number of pixels of the target region is equal to or greater than a certain ratio of the number of pixels of the target medical image.

14. The medical image processing device according to claim 2,
wherein the processor is configured to determine the certain threshold value used in a case of outputting the brightness analysis information in accordance with operation information, an imaging condition, or an imaging apparatus in a case of capturing the medical image.

15. The medical image processing device according to claim 1,
wherein the processor is configured to analyze the brightness analysis information from three or more specific medical images to select the training medical image.

16. The medical image processing device according to claim 1,
wherein the processor is configured to store the training medical image in a storage device.

17. The medical image processing device according to claim 1,
wherein the processor is configured to perform machine learning using the selected training medical image.

18. An operation method of a medical image processing device, the method comprising:
a step of acquiring a medical video image;
a step of analyzing brightness information of each of a plurality of specific medical images within a specific time range among a plurality of medical images constituting the medical video image to output brightness analysis information; and
a step of selecting a training medical image to be used for machine learning from among the plurality of specific medical images using the brightness analysis information.
